(19) United States
(12) Patent Application Publication
(10) Pub. No.: US 2020/0009282 A1
(43) Pub. Date: Jan. 9, 2020

(54) Plug in night light base

(71) Applicant: Maochen Chang, Pomona, CA (US)

(72) Inventor: Maochen Chang, Pomona, CA (US)

(21) Appl. No.: 16/026,064

(22) Filed: Jul. 3, 2018

(51) Int. Cl. A61L 9/03; F21S 8/00; F21V 33/00

(52) U.S. Cl. CPC A61L 9/03; F21S 8/035; F21V 23/04; A61L 2209/12; F21V 33/0004

(57) ABSTRACT

This invention relates in general to a device, which uses the heat from a light bulb to dispense fragrance oil. Its modern design makes it work as a functional home decor, too. It works as both a light and a warmer aid, so you get real value for what you pay for. Its color works well with any warmer color, too, making it among the most versatile of all.

### FIELD OF THE INVENTION

This invention relates in general to a device which uses the heat from a light bulb to dispense fragrance oil. More specifically, the invention provides is a simple one piece reusable unit which distributes a pleasing scent when the bulb is turned on.

### BACKGROUND OF THE INVENTION

Devices which use heat from a light bulb to dispense a fragrance or other vapor are well known in the art. There are several categories of devices which distribute scent in this manner. These categories include devices which wrap a ring of an absorbent material around the neck of the bulb, adhere an impregnated pad to the bulb, rest fragrance material in a well or depression in the top of a specially designed bulb, rest on the bulb to hold a liquid, and rest a specially formed scent impregnated polyamide resin on the bulb. For example, devices which wrap a ring of an absorbent material around the neck of the bulb are disclosed in U.S. Pat. No. 1,920,599 issued to Schuh, U.S. Pat. No. 2,238,476 issued to Monteith. U.S. Pat. No. 2,468,164 issued to Brewster.

U.S. Pat. No. 2,539,696 issued to Morrison, and U.S. Pat. No. 2,741,812 issued to Tellier. Devices which hold or adhere an impregnated pad or cake against the bulb, are disclosed in, U.S. Pat. No. 2,372,371 issued to Eisner, U.S. Pat. No 2,435,756 issued to Schlesinger, and U.S. Patent No. 4,647,433 issued to Spector. Devices which rest fragrance material in a well or depression in the top of a specially designed bulb, are disclosed in U.S. Pat. No. 1,535,486 issued to Lundy and Ratner U.S. Pat. No 4,965,490 issued to Ratner. Devices in which an attachment to the bulb holds a liquid that is vaporized by the bulb's heat are disclosed in U.S. Pat No. 1,403,548 issued to Dugerman, U.S. Pat. No. 1,556,680 issued to Dormet, U.S. Pat. No. 4,074,111 issued to Hunter, and U.S. Pat. No. 4,647,428 Gully. U.S. Pat. No. 4,184,099 issued Lindauer et al. discloses a specially formed scent impregnated polyamide resin which rests on the bulb.

All of the know prior art scent generating devices for light bulbs have had one or more disadvantages. The devices which dispense a heated liquid create a risk of lamp breakage or liquid spillage. Also personal injury could result if the hot liquid spilled on the user. Some of the devices, which around the bulb block almost of the bulb's light. Several scent producing devices are for a single use only, and when the scent is dissipated the device must be discarded.

All of the prior art inventions use gravity, a complex, molded shape, or an adhesive to hold the device against the bulb. The devices which rest around the neck of or on top of the bulb are held by gravity, so they are used only when the bulb is installed either base up or base down. The devices which fit around the entire bulb using several materials such glass, foil, or plastic plus an absorbent pad are relatively complex and expensive in construction and block a significant portion of the bulb's light. The devices which are held against the bulb by an adhesive are made of several materials and are not reusable.

In view of the above it is an object of the invention to provide an improved fragrance of safe, simple and inexpensive design that is reusable and can be utilized on a light bulb regardless of orientation of the bulb.

### SUMMARY OF THE INVENTION

The invention provides an improved fragrance dispenser of safe, simple and inexpensive design that is reusable and can be utilized on a light bulb regardless of orientation of the bulb. The fragrance dispenser securely grips the light bulb at its widest point such that the light bulb may be oriented in any direction. Thus the fragrance dispenser may be used, for example, on a telescoping desk lamp or a swinging lamp where the bulb can be oriented in any direction not merely base up or base down. The fragrance dispenser is made from absorbent material that is non-combustible and reusable, such that the absorbed oil is vaporized to scent the room air with a pleasing fragrance when the light bulb is turned on and heat is generated.

In a preferred embodiment, the fragrance dispenser includes a dish having a central opening and a plurality of slits that radiate from the central opening. The slits open to form gripping portions when the fragrance dispenser is fitted over a light bulb.

Additionally it comes with dimmer to adjust light of the lamp.

### BRIEF DESCRIPTION OF THE DRAWINGS

The invention will now be described with reference to certain preferred embodiments thereof and the accompanying drawings, wherein:

FIG. 1 is a plan view of a fragrance dispenser in accordance with the invention as a flat sheet prior to installation;

FIG. 2 is an elevational view of the fragrance dispenser being stretched over the bulb;

FIG. 3 is an elevational view of the fragrance dispenser in place around the bulb; and

FIG. 4 is an elevational view the user putting drops of fragrance oil onto the device.

### DETAILED DESCRIPTION OF THE PREFERRED EMBODIMENTS

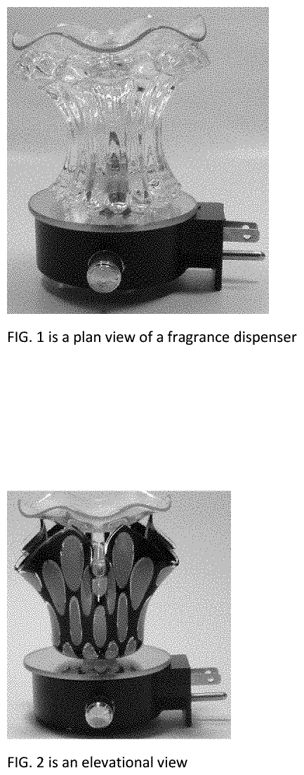

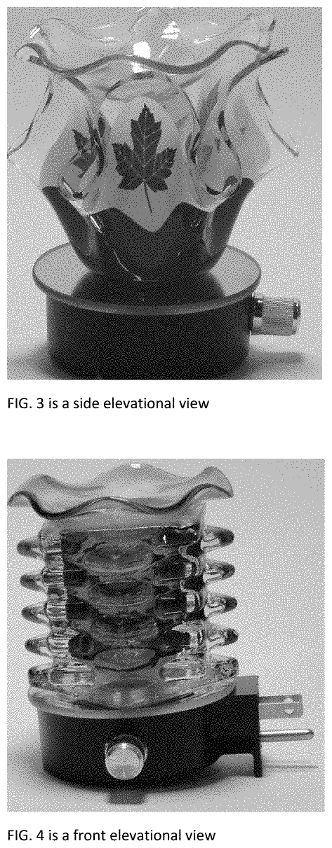

According to FIG. 1, is an incandescent lamp consisting of a tungsten filament sealed into a compact transparent envelope that is filled with a mixture of an inert gas and a small amount of a halogen such as iodine or bromine. The combination of the halogen gas and the tungsten filament produces a halogen cycle chemical reaction which redeposit's evaporated tungsten to the filament, increasing its life and maintaining the clarity of the envelope. Because of this, a halogen lamp can be operated at a higher temperature than a standard gas-filled lamp of similar power and operating life, producing light of a higher luminous efficacy and color temperature. The small size of halogen lamps permits their use in compact optical systems for projectors and illumination.

In ordinary incandescent lamps, evaporated tungsten mostly deposits onto the inner surface of the bulb, causing the bulb to blacken and the filament to grow increasingly weak until it eventually breaks. The presence of the halogen, however, sets up a reversible chemical reaction cycle with this evaporated tungsten. The halogen cycle keeps the bulb clean and causes the light output to remain almost constant throughout the bulb's life. At moderate temperatures the halogen reacts with the evaporating tungsten, the halide formed being moved around in the inert gas filling. At some point, however, it will reach higher temperature regions within the bulb where it then dissociates, releasing tungsten back onto the filament and freeing the halogen to repeat the process. The overall bulb envelope temperature must be significantly higher than in conventional incandescent lamps for this reaction to succeed.

Like all incandescent light bulbs, a halogen lamp produces a continuous spectrum of light, from near ultraviolet to deep into the infrared. Since the lamp filament can operate at a higher temperature than a non-halogen lamp, the spectrum is shifted toward blue, producing light with a higher effective color temperature and higher power efficiency. This makes halogen lamps the only option for consumer light source with black-body radiation spectrum similar to that of the Sun and most suitable for the eyes. Following invention uses halogen light to heat the oil and make smell. It comes with dimmer switch. It's convenience to plug on the wall. The entire mirror bases are the same.

As shown in FIG., a drop of fragrance oil is placed on the absorbent dish, either after or before the absorbent pad is placed on the light bulb. The user may use the fragrance oil of choice or mix several pleasing scents. Once the light bulb is turned on the absorbed fragrance oil will be vaporized due to the heat generated by the light bulb. The absorbent pad can be repeatedly recharged with oil for multiple uses.

The invention has been described with reference to certain preferred embodiment thereof. It will be understood, however, that modification and variations are possible within the scope of the appended claims. For example, the designs and gripping portions can be formed in any desired shape or configuration. Also, the absorbent pad can be presented at the time of manufacture.

1. Great for both indoor and outdoor use, this light fixture is what you need to keep your home and workspace ready for action.
Its modern design makes it work as a functional home decor, too.
It works as both a light and a warmer aid, so you get real value for what you pay for.
Its color works well with any warmer color, too, making it among the most versatile of all.
